(19) United States
(12) Patent Application Publication
(10) Pub. No.: US 2018/0154403 A1
(43) Pub. Date: Jun. 7, 2018

(54) DEVICE FOR STERILE TREATMENT

(71) Applicant: Eric Netzhammer, Arlesheim (CH)

(72) Inventor: Eric Netzhammer, Arlesheim (CH)

(21) Appl. No.: 15/737,622

(22) PCT Filed: Jun. 13, 2016
(86) PCT No.: PCT/EP2016/063436

§ 371 Date: Dec. 18, 2017

(30) Foreign Application Priority Data
Jun. 19, 2015 (CH) 00886/15

(51) Int. Cl. B08B 3/04; A61L 2/07

(52) U.S. Cl. CPC B08B 3/044; B67B 2201/08; A61L 2/07

(57) ABSTRACT

The cleaning and sterilization device is provided with a product discharge connection with a beta port that simultaneously serves to discharge the process media. To this end, a process attachment connected to the discharge connection and the process line during treatment and comprising a port opening system is provided such that the process media flow through the open port and clean and sterilize said port.

The present invention refers to a device for sterile treatment according to the preamble according to claim 1.

For transfers in the sterile technology, port systems, also known as “alfa beta ports” or “rapid transfer ports (RTP)” are used. With these systems, sterilized substances or sterilized parts may be transferred from one sterile container to another sterile container. Treatment machines for small parts (such as plug treatment machines) are normally provided with a beta port. After treatment, the treatment machine containing small parts is docked to another machine, normally a filling machine, which is provided with an alfa port. Alfa and beta ports are connected to each other, and afterwards the port door is opened in order for the transfer to take place.

In the state of the art, various treatment machines are known, which are provided with such beta ports. Systems are known, which are provided, on the upper side, i.e. on the side of the container which is up during the treatment, with two connections; one port connection for the successive transfer, and another connection for the discharge of the process media (steam/water/air/etc.) required for the treatment.

In other known systems, the container has only one connection on the upper side, which is then in turn branched into two connections, i.e. a beta port connection for the transfer after the treatment and a further connection for discharging process media.

All systems are provided with a lower connection for feeding said process media.

One of various important drawbacks of known systems is that during the treatment of the parts to be cleaned and sterilized, the beta port used for transfer has to be closed. This means that process media cannot reach certain regions on the circumference of the port lid. These regions (“ring of concern”) are thus unsterile at the end of the treatment, and may contaminate the treated parts during transfer. Devices are also known, in which the beta port is open for cleaning and wherein their critical regions are also cleaned. To this end, the containers used for treatment and transport, are attached, for cleaning, at a stationary pivoting mount, which is also provided with the feeding and discharging lines for the cleaning media.

The drawback of this solution is that a considerable additional effort is required for fixing the container to the mount and for removing the same therefrom.

The object of the invention is to avoid these drawbacks.

According to the invention, this object is achieved by the characteristics according to claim 1. An exemplary embodiment of the invention is explained in the following by means of the attached drawings. In particular

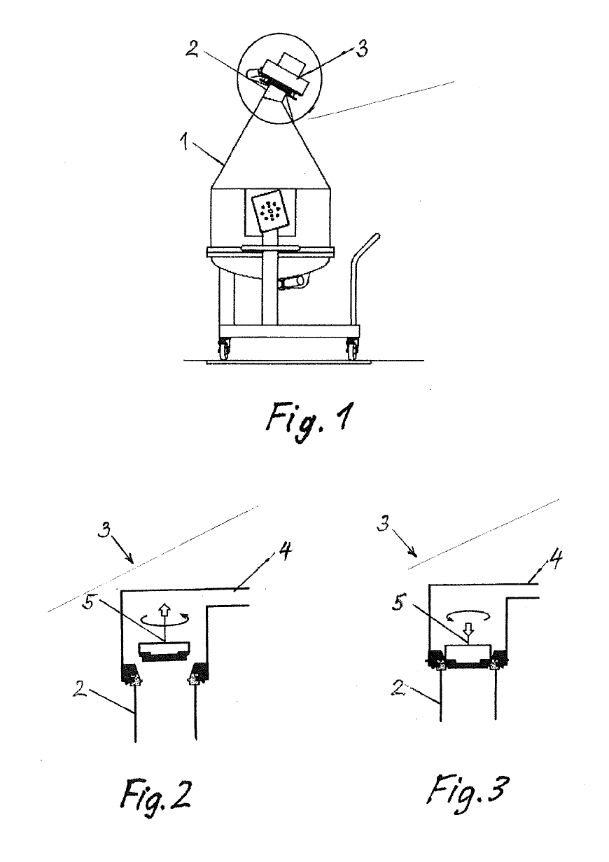

FIG. 1 shows a treatment container for small parts with only one beta port on the upper side and a process attachment,

FIG. 2 shows a schematic cross section of the process attachment with an open port,

FIG. 3 shows a schematic cross section of the process attachment with a closed port.

The present description refers to a treatment container, such as the one described in the Swiss patent application no. 01973/14, i.e. a container, which is fixedly connected to a carriage and is rotatably positioned on the carriage. As shown in FIG. 1, the treatment container 1 is provided on the upper side with only one connection 2 without branching, i.e. a beta port, which is used on one hand for the subsequent transfer and on the other hand during the treatment process, as a passage for the process media (steam/water/air/etc.).

For the treatment process, a process attachment 3 is applied onto the port and sealingly connected to the port by means of a clamping system, known per se, a screwed connection or similar. The process attachment is in turn provided with a connection 4 for the process line, through which the process media are discharged. The clamping system may be automatically or manually actuated.

As shown in FIGS. 2 and 3, inside the attachment a known port-opening-system (POS) 5 is arranged, with which, after applying the process attachment and the connection of the process line, the beta port may be opened, fulfilling any sterile requirement. The port opening system may be automatically or manually actuated.

When the port lid is open, as shown in FIG. 2, the treatment container is connected to the process line and the required media transfer may take place through the formed opening. During the passage of the process media through the container for treating the small parts, also the port lid and its seat, i.e. said critical ring of concern, is situated in the flow of process media and is cleaned and sterilized, etc. After the cleaning and sterilizing passage, the port lid may be closed again, as shown in FIG. 3, until the treatment container is docked to another container, such as the container of a filling system, for example.

1. A device for cleaning and sterilizing in a treatment container, which is fixedly connected to a carriage and which is rotatably positioned on the carriage, with a product discharge connection and a beta port, which is arranged on the discharge connection, as well as a process line for discharging process media, characterized by a process attachment connected to the discharge connection and the process line, and provided with a port opening system, such that the process media flow through the open port and clean and sterilize said port.

2. The cleaning and sterilizing device according to claim 1, characterized in that the process attachment has an inner space, through which the process media flow and in which the port opening system is positioned.

3. The cleaning and sterilizing device according to claim 1, characterized by a clamping device for sealingly connecting the process attachment onto the discharge connection.

4. The cleaning and sterilizing device according to claim 1, characterized in that the port opening system is automatically actuated.

5. The cleaning and sterilizing device according to claim 1, characterized in that the port opening system is manually actuated.

6. The cleaning and sterilizing device according to claim 3, characterized in that the clamping device is automatically actuated.

7. The cleaning and sterilizing device according to claim 3, characterized in that the clamping device is manually actuated.
